(19) United States
(12) Patent Application Publication
(10) Pub. No.: US 2024/0181146 A1
(43) Pub. Date: Jun. 6, 2024

(54) BLOOD FILTRATION SYSTEM WITH INFUSION PUMP STATE DETECTOR

(71) Applicant: Nuwellis, Inc., Eden Prairie, MN (US)

(72) Inventors: David Lerner, St. Paul, MN (US); David Haskvitz, Maple Grove, MN (US); Kalley Francis Berg, Forest Lake, MN (US); Steven D. Sandoval, Eden Prairie, MN (US)

(21) Appl. No.: 18/553,370

(22) PCT Filed: Aug. 18, 2022
(86) PCT No.: PCT/US2022/075121

§ 371 Date: Sep. 29, 2023

Related U.S. Application Data
(60) Provisional application No. 63/260,381, filed on Aug. 18, 2021.

(51) Int. Cl. A61M 1/34; A61M 5/142; A61M 5/172; A61M 39/24

(52) U.S. Cl. CPC A61M 1/3403; A61M 5/14228; A61M 5/1723; A61M 39/24; A61M 2205/3331

(57) ABSTRACT

A blood filtration system can be used with an external infusion pump and can include, among other things, a withdrawal line, an infusion (return) line, a filter coupled between the withdrawal line and the infusion line, a controller, and a withdrawal pressure sensor and/or an infusion pressure sensor. The withdrawal pressure sensor can sense a withdrawal pressure signal indicative of a pressure in the withdrawal line. The infusion pressure sensor can be coupled to the infusion line to sense an infusion pressure signal indicative of a pressure in the infusion line. In various embodiments, the controller can detect an operational state of the external infusion pump by analyzing at least the withdrawal pressure signal and/or the infusion pressure signal.

### CLAIM OF PRIORITY

This patent application claims the benefit of priority of Lerner et al., U.S. Provisional Patent Application Ser. No. 63/260,381, entitled “BLOOD FILTRATION SYSTEM WITH INFUSION PUMP STATE DETECTOR,” filed on Aug. 18, 2021, which is hereby incorporated by reference herein in its entirety.

### TECHNICAL FIELD

This document relates generally to blood filtration and more particularly, but not by way of limitation, to a blood filtration system that can detect an operational state of an infusion pump when the infusion pump is coupled to and used with the blood filtration system.

### BACKGROUND

A blood filtration system, such as an ultrafiltration (“UF”) or continuous renal replacement therapy (“CRRT”) system, can treat a patient suffering from fluid overload by removing excessive fluid and restores fluid balance. The system removes blood from the venous circulation of the patient and separates plasma water and electrolytes from erythrocytes (red blood cells) and other blood constituents using a filter (e.g., a single use, disposable filter). After filtration, some of the plasma water is conveyed to a bag for disposal. The balance of the plasma water, the erythrocytes, and other blood constituents are returned to the patient's venous circulation. As the plasma water (also referred to as “ultrafiltrate”) is removed from the circulatory system via ultrafiltration, fluid that is located in the interstitial space (e.g., tissue fluid/edema/congestion) can be recruited back into the circulatory system according to starling forces. In other words, as the plasma fluid is removed from the circulatory system, “new” fluid can translocate from the tissues back into the circulatory system, thus compensating, partially or entirely, for the recently removed fluid. The rate at which this tissue fluid flows back into the circulatory system is known as the plasma refill rate (“PRR”). This PRR depends on multiple factors including but not limited to plasma oncotic pressure, plasma hydrostatic pressure, interstitial osmotic pressure, interstitial hydrostatic pressure, and pressure derived from skin turgidity and elasticity. So, the PRR can vary from patient to patient or in one patient as therapy proceeds. If the ultrafiltration rate (“UFR”) exceeds the PRR, there can be too much plasma fluid removed from the circulatory system too quickly or in an absolute sense.

For a smaller stature patient (e.g., a patient having low body weight, a pediatric patient, or a neonatal patient) with concomitant small intravascular volumes, there is increased scrutiny of extracorporeal circulatory volume, infusion rates, and ultrafiltration rates. Movement of excessive blood volume from the intracorporeal to extracorporeal circulation can lead to hemodynamic instability. There are multiple mechanisms of action relating to this phenomenon of hemodynamic instability including, but not limited to, utilization of a too rapid extracorporeal blood flow rate, excessive thermal energy transfer, too rapid removal of plasma water leading to inadequate organ system and tissue perfusion, among others.

Additionally, exogenous substances (e.g., anticoagulants, nutrition, medications, and/or saline) are often infused into the patient simultaneously with ultrafiltration and/or CRRT modalities. These substances can be infused into the patient using an infusion pump system while the blood filtration system is removing fluid from the patient. The blood filtration system and the infusion pump system can be separate and distinct systems that do not communicate with each other (e.g., electrical signals are not transmitted between the blood filtration system and the infusion pump system, or the like). Operation of the blood filtration system needs to be controlled for patient safety given all these factors affecting hemodynamic stability.

### SUMMARY

A blood filtration system can be used with an external infusion pump and can include, among other things, a withdrawal line, an infusion (return) line, a filter coupled between the withdrawal line and the infusion line, a controller, and a withdrawal pressure sensor and/or an infusion pressure sensor. The withdrawal pressure sensor can sense a withdrawal pressure signal indicative of a pressure in the withdrawal line. The infusion pressure sensor can be coupled with the infusion line to sense an infusion pressure signal indicative of a pressure in the infusion line. In various embodiments, the controller can detect an operational state of the external infusion pump by analyzing at least the withdrawal pressure signal and/or the infusion pressure signal.

In one embodiment, a blood filtration system can be coupled with an external infusion pump. For instance, the blood filtration system includes a catheter, and the system is coupled to a patient through the catheter. For instance, the catheter can include a blood circuit having a catheter. The catheter can be inserted into vasculature of a patient, and the system can pump blood from the vasculature and through the blood circuit. The blood filtration system can use a filter to reduce one or more plasma constituents in blood of the patient. For example, the system removes blood from the venous circulation of the patient and pumps the blood through a blood circuit (e.g., an extracorporeal blood circuit, or the like). Accordingly, the blood circuit can communicate fluid with the vasculature of the patient, for instance to reduce one or more plasma constituents in the blood of the patient.

The system can include a withdrawal line configured to be coupled to the catheter to receive the blood from the patient through the catheter. In another example, an infusion line can be coupled to the catheter to return filtered blood into the patient through the catheter. In yet another example, the system can include an infusion port in fluidic communication with one or more of the withdrawal line or the infusion line. The infusion port can be coupled to the external infusion pump, and the blood filtration system can receive an infusion fluid from the external infusion pump at the infusion port. The system can include a filter configured to be coupled between the withdrawal line and the infusion line and to produce the filtered blood by removing portions of the one or more plasma constituents in the blood. In still yet another example, the blood filtration system can include a blood pump to pump the blood through the system, for instance by pumping blood through the filter at an extracorporeal blood flow rate (“EBFR”). In a further example, a filtration pump removes a filtrate fluid including the removed portions of the one or more plasma constituents from the filter at an ultrafiltration rate. The system can include a controller, and the controller can include one or more of a signal analyzer and an infusion pump state detector. The signal analyzer can analyze at least one of the withdrawal pressure signal or the infusion pressure signal. The infusion pump state detector can detect an operational state of the external infusion pump using an outcome of the analysis.

In one embodiment, a method for operating a blood filtration system is provided. The blood filtration system is coupled to a patient through a catheter to reduce one or more plasma constituents in blood of the patient and coupled to an external infusion pump for infusing an infusion fluid to the patient through the blood filtration system. The method can include: pumping blood through a withdrawal line coupled to the catheter, a filter coupled to the withdrawal line, and an infusion line coupled between the filter and the catheter at an extracorporeal blood flow rate (EBFR) using a blood pump engaged with the withdrawal line; pumping the infusion fluid into the withdrawal line or the infusion line from the external infusion pump; removing portions of the one or more plasma constituents in the blood using the filter; removing a filtrate fluid including the removed portions of the one or more plasma constituents from the filter at an ultrafiltration rate (UR) using a filtration pump; sensing at least one of a withdrawal pressure signal indicative of a pressure in the withdrawal line or an infusion pressure signal indicative of a pressure in the infusion line; and detecting an operational state of the external infusion pump by analyzing the at least one of the withdrawal pressure signal or the infusion pressure signal.

This summary is an overview of some of the teachings of the present application and not intended to be an exclusive or exhaustive treatment of the present subject matter. Further details about the present subject matter are found in the detailed description and appended claims. The scope of the present invention is defined by the appended claims and their legal equivalents.

### BRIEF DESCRIPTION OF THE DRAWINGS

FIG. 1 is an illustration of an embodiment of a blood filtration system coupled to an external infusion pump.

FIG. 2 is a block diagram illustrating an embodiment of the external infusion pump and portions of the blood filtration system of FIG. 1 related to detection of an operational state of the external infusion pump.

FIG. 3A is a graph showing an example of a pressure signal created by an infusion pump, such as the external infusion pump shown in FIGS. 1 and 2.

FIG. 3B is a graph showing a Fast Fourier Transform (FFT) of the pressure signal of FIG. 3A.

FIG. 4A is a graph showing an example of a pressure signal created by a blood pump, such as the blood pump shown in FIGS. 1 and 2.

FIG. 4B is a graph showing a Fast Fourier Transform of the pressure signal of FIG. 4A.

FIG. 5A is a graph showing an example of the sum of pressure signals created by a blood pump and an infusion pump, such as the blood pump and the external infusion pump shown in FIGS. 1 and 2.

FIG. 5B is a graph showing a Fast Fourier Transform of the sum of the pressure signals of FIG. 5A.

FIG. 6A is a graph showing an example of a random noise signal.

FIG. 6B is a graph showing a Fast Fourier Transform of the random noise signal of FIG. 6A.

FIG. 7A is a graph showing an example of the sum of pressure signals created by a blood pump and an infusion pump, such as the blood pump and the external infusion pump shown in FIGS. 1 and 2, and random noise.

FIG. 7B is a graph showing a Fast Fourier Transform of the sum of the pressure signals and the random noise of FIG. 7A.

FIG. 8A is a graph showing an example of a pressure signal, such as a pressure signal created by the blood pump and/or the external infusion pump shown in FIGS. 1 and 2.

FIG. 8B is a graph showing an autocorrelation of the pressure signal of FIG. 8A.

FIG. 9A is a graph showing an example of a random noise signal.

FIG. 9B is a graph showing an autocorrelation of the random noise signal of FIG. 9A.

FIG. 10A is a graph showing an example of the sum of a pressure signal, such as a pressure signal created by the blood pump and/or the external infusion pump shown in FIGS. 1 and 2, and a random noise signal.

FIG. 10B is a graph showing an autocorrelation of the sum of the pressure and random noise signals of FIG. 10A.

FIG. 11 is a circuit diagram illustrating an electrical circuit model for the pneumatic connections of an external infusion pump, a blood pump, a catheter, and a blood filtration system, such as in the setup illustrated in FIG. 1.

FIG. 12 is a block diagram illustrating an embodiment of a controller of a blood filtration system, such as the controller shown in FIGS. 1 and 2.

FIG. 13 is a flowchart illustrating an embodiment of a method for operating a blood filtration system coupled to an external infusion pump.

FIG. 14 is a flowchart illustrating an embodiment of a method for operating the blood filtration system while monitoring on-off state of the external infusion pump periodically.

FIG. 15 is an illustration of another embodiment of the blood filtration system coupled to the external infusion pump.

### DETAILED DESCRIPTION

The following detailed description of the present subject matter refers to subject matter in the accompanying drawings which show, by way of illustration, specific aspects and embodiments in which the present subject matter can be practiced. These embodiments are described in sufficient detail to enable those skilled in the art to practice the present subject matter. References to “an”, “one”, or “various” embodiments in this disclosure are not necessarily to the same embodiment, and such references contemplate more than one embodiment. The following detailed description is demonstrative and not to be taken in a limiting sense. The scope of the present subject matter is defined by the appended claims, along with the full scope of legal equivalents to which such claims are entitled.

This document discusses, among other things, a blood filtration system, such as an ultrafiltration (UF) or continuous renal replacement therapy (CRRT) system, that can be used with an infusion pump and controlled based on an operational state of the infusion pump. The blood filtration system can detect the operational state of the infusion pump and control its own operation accordingly, for example by adjusting the ultrafiltration rate (UFR) to maintain a safe net fluid removal rate. In this document, the infusion pump is referred as an “external infusion pump” because it can be considered to be a device that is external to the blood filtration system. For example, the external infusion pump and the blood filtration system can be two separate products that can be supplied by the same manufacturer or different manufacturers. In another example, electrical signals are not transmitted between the external infusion pump and the blood filtration system, For instance, the blood filtration system does not receive information (e.g., data, digital signals, analog signals, or the like) from the external infusion pump regarding the operational state of the external infusion pump. The present subject matter allows the blood filtration system to detect the operational state of the external infusion pump and take the detected operational state into account when controlling its own operation without direct (e.g., electronic) communication with the external infusion pump.

In this document, unless noted otherwise, a “patient” includes a person receiving treatment using the blood filtration system and the external infusion pump. A “user” includes a physician or other clinician who participates in the treatment of the patient using the blood filtration system and the external infusion pump.

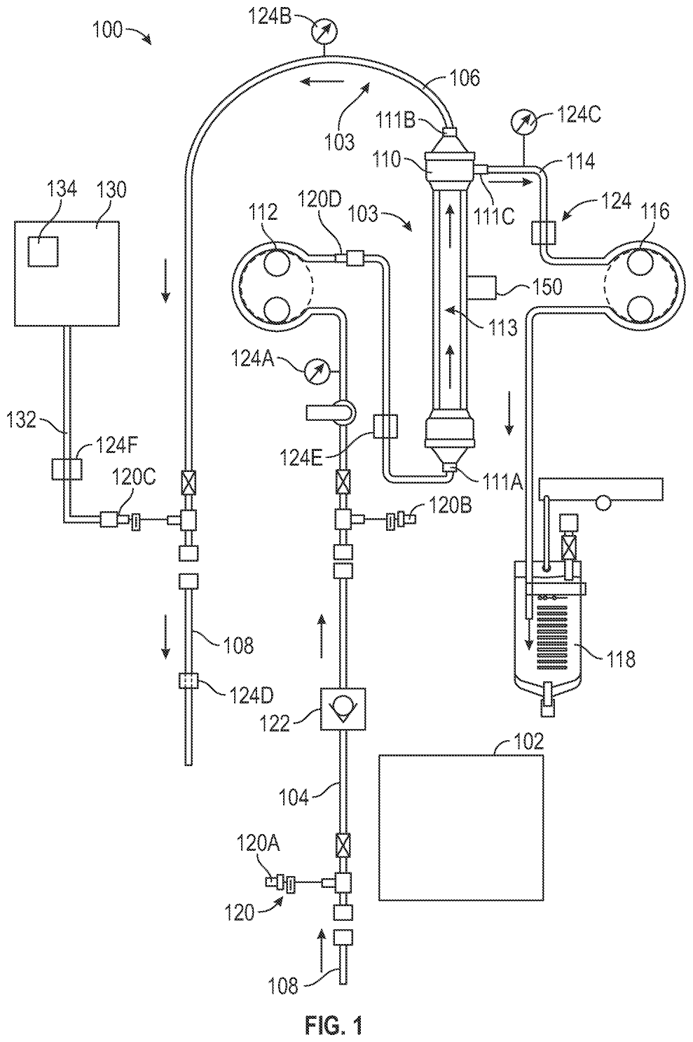

FIG. 1 is an illustration of an embodiment of a blood filtration system 100 coupled to an external infusion pump 130. Blood filtration system 100 and external infusion pump 130 can be separate, independently controlled products without direct electronic communication with each other. Blood filtration system 100 and external infusion pump 130 are illustrated and discussed as a non-limiting example of a system in which the present subject matter can be applied. In various embodiments, the blood filtration system and the external infusion pump according to the present subject matter can include some or all of the components shown in FIG. 1, as well as components not shown in FIG. 1, as understood by those skilled in the art.

Blood filtration system 100 can reduce one or more plasma constituents (e.g., water, proteins, electrolytes, or the like) in blood of a patient. Blood filtration system 100 includes a controller 102. Controller 102 can include processing circuitry that can control one or more components of blood filtrations system 100. In various embodiments, the electronic circuits of the blood filtrations system 100 can be implemented using a combination of hardware and software. For example, the circuit of controller 102, including its various embodiments discussed in this document, can be implemented using an application-specific circuit constructed to perform one or more particular functions and/or a general-purpose circuit programmed to perform such function(s). Such a general-purpose circuit includes, but is not limited to, a microprocessor or a portion thereof, a microcontroller or portions thereof, and a programmable logic circuit or a portion thereof.

In an example, the blood filtration system 100 includes a blood circuit 103. The blood circuit 103 can transport blood through the system 100. In the illustrated embodiment, blood circuit 103 includes a withdrawal line 104 and an infusion line (also referred as return line) 106. Lines 104 and 106 can each be coupled with a catheter 108 and can transmit blood within blood filtration system 100. Catheter 108 can be inserted into a blood stream of the patient. For example, catheter 108 can be inserted into the basilic vein, the cephalic vein, brachial vein, the axillary vein, the subclavian vein, the brachiocephalic vein, or the like. Blood can flow from the blood stream of the patient into catheter 108, into withdrawal line 104, through other components of blood filtration system 100, through infusion line 106, into catheter 108, and back into the blood stream. Catheter 108 can represent one or more catheters. In one embodiment, catheter 108 is a single catheter including a withdrawal lumen in fluidic communication with withdrawal line 104 and an infusion lumen in fluidic communication with infusion line 106 when lines 104 and 106 are both coupled to catheter 108.

A filter 110 is coupled between withdrawal line 104 and infusion line 106. For example, lines 104 and 106 can include one or more fittings that facilitate the coupling to filter 110. In the illustrated embodiment, filter 110 includes a filter inlet port 111A coupled to withdrawal line 104 and a filter outlet port 111B coupled to infusion line 106. Filter 110 can reduce portions of one or more plasma constituents (e.g., water, electrolytes, or the like) in the blood flowing through it while producing a filtrate fluid including the one or more plasma constituents removed from the blood. Filter 110 has a base filter fluid capacity 113.

Blood filtration system 100 includes a blood pump 112 that can pump (e.g., convey, drive, push, or the like) blood through blood circuit 103. In the illustrated embodiment, blood pump 112 is a peristaltic pump and engages with withdrawal line 104 to pump blood through withdrawal line 104 and into filter 110. Controller 102 can control a speed of blood pump 112, referred as the extracorporeal blood flow rate (EBFR), which is the flow rate of blood through blood circuit 103. The blood pump 112 can include a syringe pump.

Blood filtration system 100 includes a filtration line 114 and a filtration pump 116. Filter 110 includes a filtrate fluid port 111C coupled to filtration line 114 and can transmit the filtrate fluid to filtration line 114 through filtrate fluid port 111C. Filtration pump 116 can receive the filtrate fluid from filtration line 114 and pump the filtrate fluid into a filtrate fluid reservoir 118 (e.g., a bag, container, bladder, or the like). In the illustrated embodiment, filtration pump 116 is a peristaltic pump that engages with filtration line 114 to pump the filtrate fluid through filtration line 114. Controller 102 can control a speed of filtration pump 116, referred to as the ultrafiltration rate (UR), which is the flow rate of filtrate fluid in filtration line 114.

In the illustrated embodiment, blood filtration system 100 includes access ports 120, including a first access port 120A, a second access port 120B, and a third access port 120C. In various embodiments, blood filtration system 100 can include any one or more of such access ports. Access ports 120 allow for extraction of blood from blood filtration system 100 and/or injection of substances into the blood within blood filtration system 100. In the illustrated embodiment, access ports 120A and 120B are in communication with withdrawal line 104, and access port 120C is in communication with infusion line 106.

A valve 122 (e.g., a mechanical check valve, electronically controlled valve, or the like) can be positioned between access ports 120A and 120B and configured to allow the blood to flow unidirectionally within withdrawal line 104 (e.g., unidirectionally from catheter 108 to filter 110). This allows a substance to be injected into withdrawal line 104 at access port 120B and allows blood without the injected substance to be withdrawn from access port 120A. For example, a blood thinner (e.g., heparin, or the like) can be infused into the blood through access port 120B, and blood is drawn through access port 120A for measuring blood clotting time parameters of the patient. Because the blood drawn through access port 120A does not include the blood thinner infused through access port 120B, the blood clotting time parameter determination is not affected by the blood thinner injection.

Blood filtration system 100 can include sensors 124 (e.g., pressure transducer, accelerometer, or the like). A first sensor 124A can be a pressure sensor that measures (e.g., determine, calculate, obtain, provide, or the like) a pressure within withdrawal line 104, a second sensor 124B can be a pressure sensor that measures the pressure within infusion line 106, and the third sensor 124C can be a pressure sensor that measures the pressure within filtration line 114. Sensors 124 can also include a fourth sensor 124D (e.g., a position sensor, or the like) and a fifth sensor 124E (e.g., blood flow rate sensor, or the like). Sensor 124E can measure the blood flow rate through blood filtration system 100 (e.g., as represented by the blood flow rate in withdrawal line 104, as illustrated in FIG. 1).

Sensors 124 can also include a gas detector that detects a discontinuity in flow of liquid through blood circuit 103. For example, sensor 124E can be a gas detector that can detect air or other gases in blood circuit 103, and sensor 124F can be a gas detector that can detect air or other gases an external infusion line 132. In the illustrated embodiment, sensor 124E is located between blood pump 112 and filter 110 and configured to provide a signal to controller 102 in response to detecting a discontinuity in flow of liquid in withdrawal line 104. Controller 102 can stop blood pump 112 and/or filtration pump 116 in response to receiving the signal provided by sensor 124E. Thus, blood filtration system 100 can prevent gas (e.g., an air bubble, or the like) from entering vasculature of the patient.

Sensors 124 can also include one or more hematocrit sensors each configured to determine a hematocrit of the blood following through blood circuit 103 (e.g., in withdrawal line 104 and/or infusion line 106). Hematocrit is the ratio of the volume of red blood cells to the total volume of the blood.

External infusion pump 130 can be coupled to blood filtration system 100 to pump an infusion fluid into the patient through blood circuit 103. The infusion fluid can include saline, heparin, electrolytes, and/or the like. External infusion pump 130 can be in fluidic communication with the blood circuit 103 through external infusion line 132 coupled to one of access ports 120 used as an infusion port. In the illustrated embodiment, infusion pump 130 can pump the infusion fluid into infusion line 106 though external infusion line 132 and access port 120C. In another embodiment, infusion pump 130 can pump the infusion fluid into withdrawal line 104 though external infusion line 132 and access port 120B. Accordingly, the infusion port can communicate fluid to the blood circuit.

Blood filtration system 100 can cooperate with external infusion pump 130 to remove portions of the one or more plasma constituents form the blood of the patient while maintaining hemodynamic stability of the patient. Filtration pump 116 can pump the extracted filtrate fluid from filter 110. External infusion pump 130 can pump the infusion fluid (also referred to as replacement fluid) into the blood circuit 103, for example to replace a specified quantity (e.g., a portion or an entirety) of the filtrate fluid extracted from filter 110. Replacing the filtrate fluid with the infusion fluid can facilitate clearance of the one or more plasma constituents from the blood of the patient. The filtrate fluid can include, for example, water, electrolytes, and urea. The infusion fluid can include water and electrolytes (without urea). Replacing the filtrate fluid with the infusion fluid can facilitate clearance of plasma constituents (e.g., urea or the like) because the plasma constituents are removed in the filtrate fluid and not replaced by the infusion fluid. In other words, replacing the filtrate fluid with the infusion fluid can facilitate maintenance of hemodynamic stability of the patient because the infusion fluid replaces the water and electrolytes extracted by filter 110.

External infusion pump 130 is external to (e.g., not included in, separate from, distinct from, or the like) the blood filtration system 100. For example, external infusion pump 130 cannot communicate with, or otherwise cannot be controlled by, controller 102 of blood filtration system 100. External infusion pump 130 includes an infusion pump controller 134 that can control operation of external infusion pump 130. Controller 102 and controller 134 can function independently, without direct communication (e.g., not through a user, not through an electrical communication pathway for instance wires or wirelessly, or the like) with each other. In another example, electrical signals are not transmitted between the external infusion pump and the blood filtration system, For instance, the blood filtration system does not receive information (e.g., data, digital signals, analog signals, or the like) from the external infusion pump regarding the operational state of the external infusion pump. However, the maintenance of hemodynamic stability of the patient requires coordinated operations of external infusion pump 130 and blood filtration system 100.

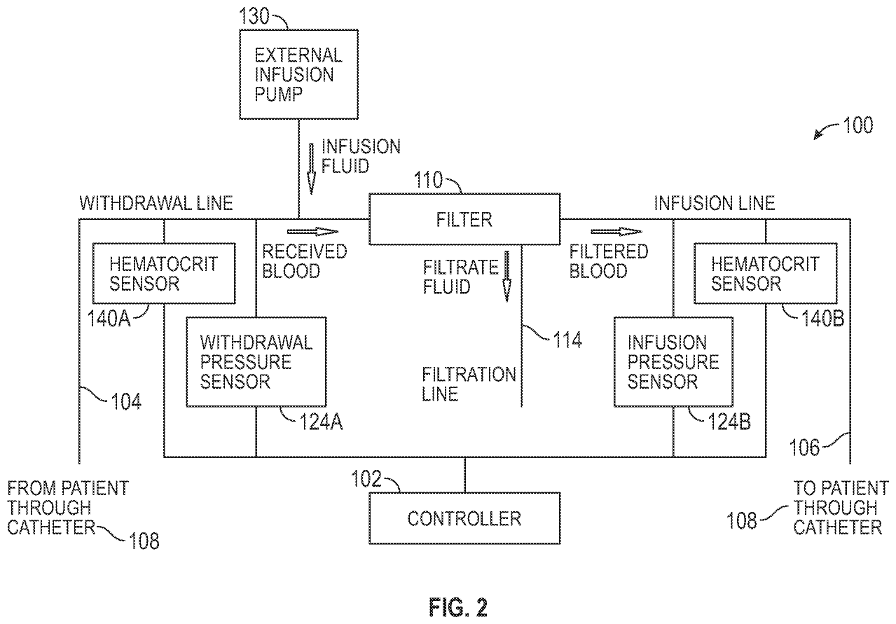

FIG. 2 is a block diagram illustrating an embodiment of external infusion pump 130 and portions of blood filtration system 100 that are related to detection of an operational state of external infusion pump 130 for coordinating operations of external infusion pump 130 and blood filtration system 100. The portions of blood filtration system 100 shown in FIG. 2 include withdrawal line 104, filter 110, infusion line 106, a withdrawal pressure sensor 124A, an infusion pressure sensor 124B, a hematocrit sensor 140A, a hematocrit sensor 140B, and controller 102. Withdrawal pressure sensor 124A, infusion pressure sensor 124B, hematocrit sensor 140A, and hematocrit sensor 140B are each an instance of sensors 124. Withdrawal pressure sensor 124A can sense a withdrawal pressure signal indicating a pressure in withdrawal line 104. Infusion pressure sensor 124B can sense an infusion pressure signal indicating a pressure in infusion line 106. Hematocrit sensor 140A can sense a hematocrit signal representing a hematocrit of the blood in withdrawal line 104. Hematocrit sensor 140B can sense a hematocrit signal representing a hematocrit of the blood in infusion line 106. In various embodiments, blood filtration system 100 can include any one or any combination of withdrawal pressure sensor 124A, infusion pressure sensor 124B, hematocrit sensor 140A, hematocrit sensor 140B, depending on how controller 102 detects the operational state of external infusion pump 130 as discussed in this document.

As illustrated in FIG. 2, the infusion fluid is pumped into filter 110 from external infusion pump 130. Simultaneously, the filtrate fluid is removed to filtration line from filter 110. Thus, there is an unknown net fluid removal (e.g., difference between the volume of the infusion fluid infused and the volume of the filtrate fluid removed) from the patient. For both adult and non-adult patients, the unknown net fluid removal can pose a risk as the true net fluid removal rate and volume are not known unless the user maintains knowledge of parameters of both blood filtration system 100 and external infusion pump 130.

Another concern arises when the user adjusts the fluid removal rate (e.g., the UR) to account for the simultaneous infusion. For example, it can occur that the delicate balance the user is trying to achieve is upset when external infusion pump system 130 is turned off for any reason (e.g., the infusion fluid bag is empty, air bubbles are detected, or the like). The net fluid removal will increase if the fluid removal by blood filtration system 100 continues while the fluid infusion by external infusion pump 130 is stopped. There is a need to alert blood filtration system 100 to stop the fluid removal when external infusion pump system 130 is turned off. Otherwise, the patient can become hypovolemic with consequences for hemodynamic instability (e.g., hypotension, loss of perfusion, or the like).

Therefore, it would be advantageous for blood filtration system 100 to be able to monitor the operational state (e.g., on or off, infusion rate, or the like) of external infusion pump 130. One approach to such monitoring includes establishing electronic communication between blood filtration system 100 and external infusion pump 130. For example, flow and volume sensors can be applied to external infusion pump 130 with sensed information communicated to blood filtration system 100. However, in an example, inter-device/system communication between blood filtration system 100 and external infusion pump 130 can not be possible or feasible, for example, when blood filtration system 100 and external infusion pump 130 are separate products that can be provided by different manufacturers. Accordingly, the blood system 100 can function independently, without direct communication with, the external infusion pump 130.

The present subject matter uses one or more sensors within blood filtration system 100 to monitor the operational state of external infusion pump 130. In various embodiments, blood filtration system 100 can uses multiple modalities in a hybrid approach to detect whether external infusion pump 130 is infusing the infusion fluid into blood circuit 103 (e.g., withdrawal line 104 or infusion line 106). In various embodiments, the detection can be performed by using sensors already provided in a product implementing blood filtration system 100, without the need for additional flow, pressure, and/or volume sensors. The present subject matter can be applied to maintain appropriate fluid balance and to prevent hypovolemia and concomitant hemodynamic instability.

In various embodiments, blood filtration system 100 can monitor the operational state of external infusion pump 130 using one or more pressure signals and/or one or more hematocrit signals. The one or more pressure signals can be sensed by pressure sensor(s) 124A and/or 124B. The one or more hematocrit signals can be sensed by hematocrit sensor(s) 140A and/or 140B. Which signal(s) to be used can depend on availability of each of sensors 124A, 124B, 140A, and 140B in each instance of blood filtration system 100 as a product and a desired level of detection reliability. For example, if blood filtration system 100 includes one of pressure sensors 124A and 124B, controller 102 can detect the operational state of external infusion pump 130 using the pressure signal sensed by pressure sensor 124A or 124B, whichever exists. If blood filtration system 100 includes both pressure sensors 124A and 124B, controller 102 can detect the operational state of external infusion pump 130 using the pressure signals sensed by pressure sensors 124A and 124B. If blood filtration system 100 further includes at least one of hematocrit sensor 140A or 140B, controller 102 can detect the operational state of external infusion pump 130 using the hematocrit signal sensed by hematocrit sensor 140A or 140B in addition to the pressure signals sensed by pressure sensors 124A and 124B. In various embodiments, controller 102 can analyze the pressure signal(s) and/or the hematocrit signal(s) and to detect the operational state of external infusion pump 130 using an outcome of the analysis. Examples of the signal analysis and the infusion pump operational state detection are discussed as follows.

### Detection Using Pressure Signal(s)

### (a) Fourier Transform of Pressure Signal

External infusion pump 130 can be coupled to blood circuit 103 at either withdrawal line 104 (e.g., as illustrated in FIG. 2) or infusion line 106 (e.g., as illustrated in FIG. 1). In either case, blood filtration system 100 includes a withdrawal pressure sensor 124A to measure the withdrawal pressure signal (indicating the pressure at the inlet of filter 110) and/or infusion pressure sensor 124B to measure the infusion pressure signal (indicating the pressure at the outlet of filter 110). When external infusion pump 130 is in an on-state and the infusion fluid is flowing into blood circuit 103, a pressure signal is created in blood circuit 103, over and above a pressure signal that is created in blood circuit 103 by blood pump 112. These pressure signals can be sensed by each of pressure sensors 124A and 124B. In the time domain, the pressure signal created by external infusion pump 130 can be difficult to detect from the sensor signal produced by pressure sensor 124A or 124B that also includes the pressure signal created by blood pump 112, the venous circulation, and patient movement. On the other hand, the pressure signal created by external infusion pump 130 can be detected in the frequency domain using a Fourier or other Transform of the pressure signal. Various methods for producing the Fourier Transform are available. The basic Discrete Fourier Transform (“DFT”), which transforms a sequence of complex numbers {xₙ}:=x₀, x₁ . . . x_{N-1} to another sequence of complex numbers {Xₖ}:=X₀, X₁ . . . X_{N-1} can be defined by the formula:

$${X}_{k} = {\sum }_{n = 0}^{N - 1} {x}_{n} {e}^{- i 2 π kn / N} k = 0 , … , N - 1 ,$$

Fast Fourier Transform (“FFT”) can be used to compute the DFT.

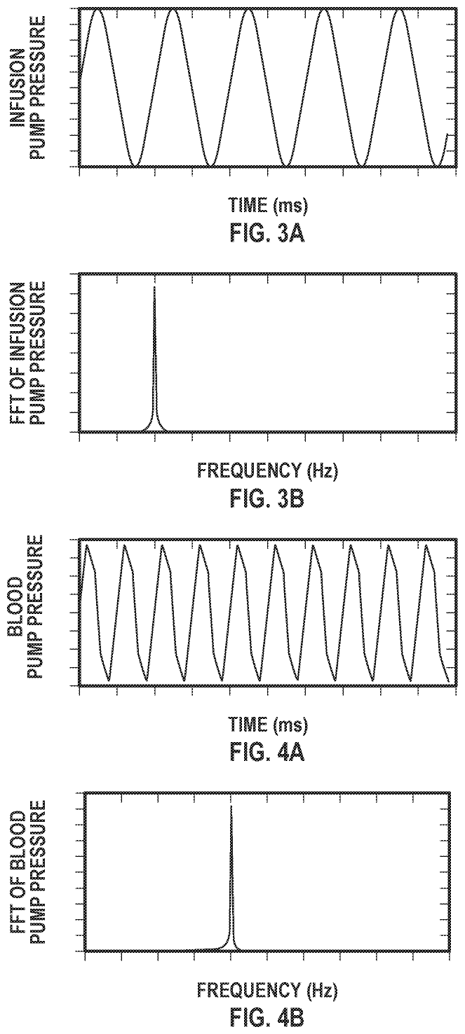

FIG. 3A is a graph showing an example of a pressure signal created by an infusion pump, such as external infusion pump 130, and FIG. 3B is a graph showing an FFT of that pressure signal. FIG. 4A is a graph showing an example of a pressure signal created by a blood pump, such as blood pump 112, and FIG. 4B is a graph showing an FFT of that pressure signal. In the frequency domain (FIGS. 3B and 4B), the pressure signals created by external infusion pump 130 and blood pump 112 are each seen as a spike at the frequency of operation of the respective pump.

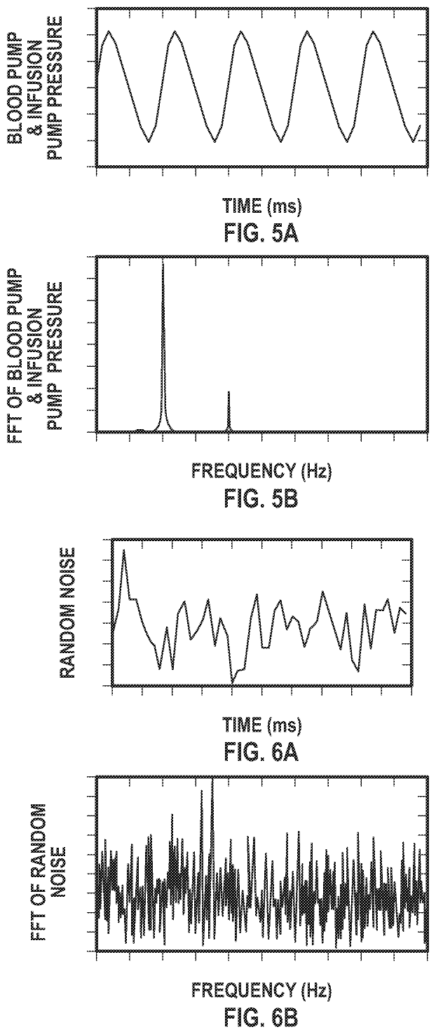

FIG. 5A is a graph showing an example of the sum of pressure signals (with different magnitudes and frequencies) created by a blood pump and an infusion pump, such as blood pump 112 and external infusion pump 130, and FIG. 5B is a graph showing a Fast Fourier Transform of the sum of these pressure signals. In the time domain (FIG. 5A), it is impossible to detect each of the two pressure signals from the signal being their sum. In the frequency domain (FIG. 5B), each signal are clearly shown as a separate spike.

FIG. 6A is a graph showing an example of a random noise signal, and FIG. 6B is a graph showing an FFT of that random noise signal. The random noise signal simulates noises due to patient motion, venous circulation, and noise in the electronics coupled to the pressure sensor for processing the sensed pressure signal. No distinct spike is seen in the frequency domain (FIG. 6B).

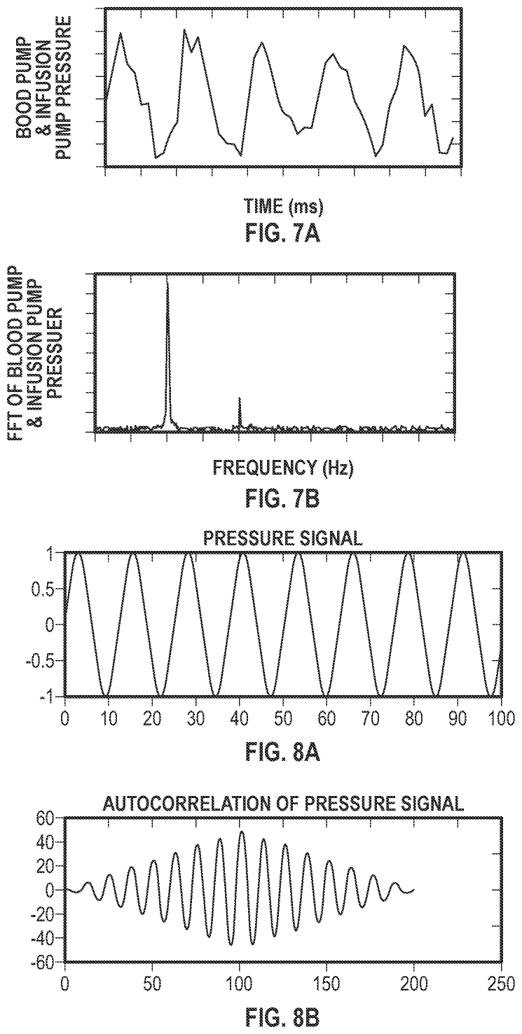

FIG. 7A is a graph showing an example of the sum of pressure signals created by a blood pump and an infusion pump, such as blood pump 112 and external infusion pump 130, and random noise, and FIG. 7B is a graph showing an FFT of the sum of the pressure signals and the random noise. In the time domain (FIG. 7A), it is impossible to it is impossible to detect each of the two pressure signals from the signal being the sum of the two pressure signals and random noise. In the illustrated example, the time domain signal appears as only the dominant signal (e.g., the pressure signal created by the blood pump, or the like). In the frequency domain (FIG. 7B), the pressure signal created by blood pump 112 and the pressure signal created by external infusion pump 130 can each be clearly seen as a spike at the frequency of operation of the respective pump.

Because each spike in the frequency domain is present only when the pump which creates the pressure signal is in an on-state, the spike can be used to detect the on/off state of the pump. This allows the on/off state of external infusion pump 130 to be detected based on the presence or absence of the spike in the frequency domain. In various embodiments, the detection can be performed automatically (e.g., by configuring controller 102 to perform the detection, or the like) or manually by the user. In various embodiments, blood filtration system 100 can be periodically turned off for a brief period of time. During this brief period of time, the spike created by blood pump 112 will vanish, leaving only the spike created by external infusion pump 130 if it is in the on-state.

### (b) Autocorrelation of Pressure Signal

Another approach to addressing the difficulty of detecting the pressure signal created by external infusion pump 130 from the sensor signal produced by pressure sensor 124A or 124B, which also includes the pressure signal created by blood pump 112 and random noise as discussed above, in the time domain is to produce an autocorrelation of the sensor signal. An autocorrelation function can be performed on the signal being the sum of the pressure signal created by a blood pump (e.g., blood pump 112), the pressure signal created by an infusion pump (e.g., external infusion pump 130), and random noise. The autocorrelation allows non-random signals (e.g., signals characteristic of pressure created by pumps) to be distinguished from random signals. Autocorrelation is a statistical function in which is the correlation of a signal with a delayed copy of itself as a function of delay. The autocorrelation function between times t₁ and t₂ can be defined by the formula:

$${R}_{XX} ( {t}_{1} , {t}_{2} ) = E [ {X}_{{t}_{1}} {\overline{X}}_{{t}_{2}} ]$$

where E is the expected value operator, and the bar represents complex conjugate.

FIG. 8A is a graph showing an example of a pressure signal (simulated as a non-random signal), such as a pressure signal created by blood pump 112 and/or external infusion pump 130, and FIG. 8B is a graph showing an autocorrelation of that pressure signal. It is seen in FIG. 8B that as the pressure signal approaches perfect overlap with itself, the autocorrelation amplitude increases and then decreases. When there is zero phase difference (delay) between the signal and itself, the autocorrelation is at a maximum.

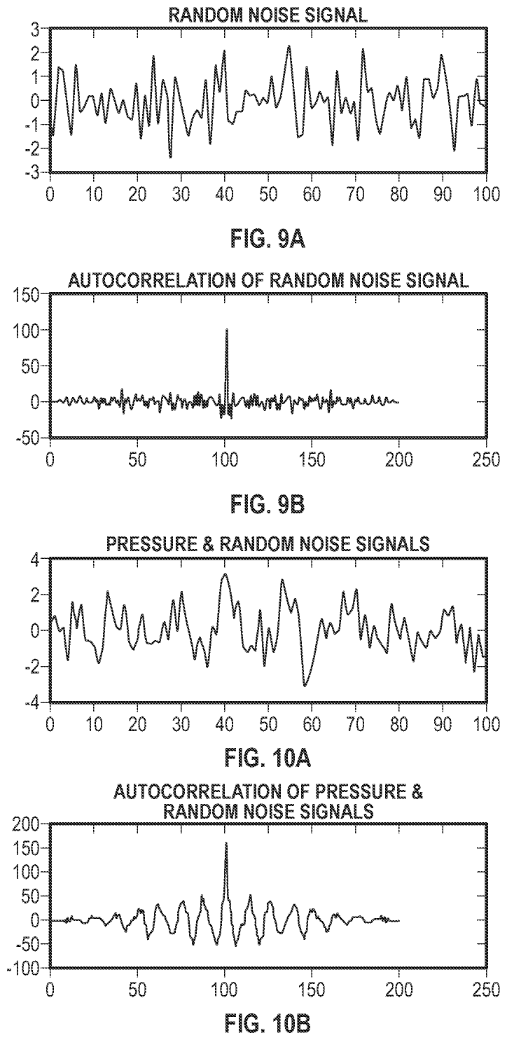

FIG. 9A is a graph showing an example of a random noise signal, and FIG. 9B is a graph showing an autocorrelation of that random noise signal. In contrast to the non-random signal shown in FIGS. 8A and 8B, the autocorrelation signal shown in FIG. 9B rises above a noise floor only when the time domain signal is at zero phase with respect to itself and is near zero at every other point. Thus, periodic signals show a gradual increase and then decrease in autocorrelation, while random signals show an increased autocorrelation amplitude only at zero phase. This characteristic of periodic signals can be used to detect the on/off state of external infusion pump 130.

FIG. 10A is a graph showing an example of the sum of a pressure signal, such as a pressure signal created by blood pump 112 and/or external infusion pump 130, and a random noise signal, and FIG. 10B is a graph showing an autocorrelation of the sum of these pressure and random noise signals. The characteristic gradual increase and decrease in amplitude as shown in FIG. 10B demonstrates the presence of a non-random signal that is not readily apparent in the time domain signal shown in FIG. 10A.

In various embodiments, blood filtration system 100 can be periodically turned off for a brief period of time. During this brief period of time, the autocorrelation indicates presence of a periodic signal if external infusion pump is in an on-state and does not indicate presence of a periodic signal (e.g., showing a random signal) if external infusion pump is in an off-state.

In various embodiments, autocorrelation can be used to detect the on/off state of external infusion pump 130 alone or in combination with the Fourier Transform. For example, when better reliability (e.g., higher sensitivity to changes in the detection of the operational state of external infusion pump 130) is desired, detection based on the autocorrelation can be used as an orthogonal approach to complement the detection based on the Fourier Transform.

### (c) Change in Pressure Signal Due to Blood Pump Pause

The operational state of external infusion pump 130 can also be detected using a change in the withdrawal pressure (e.g., sensed using withdrawal pressure sensor 124A) or the infusion pressure (e.g., sensed using infusion pressure sensor 124B) that results from a change in the operational state of blood pump 112. For example, blood pump 112 can be turned off briefly, and the change in the withdrawal pressure or the infusion pressure resulting from this blood pump pause can be analyzed to determine whether external infusion pump 130 is in the on-state or off-state.

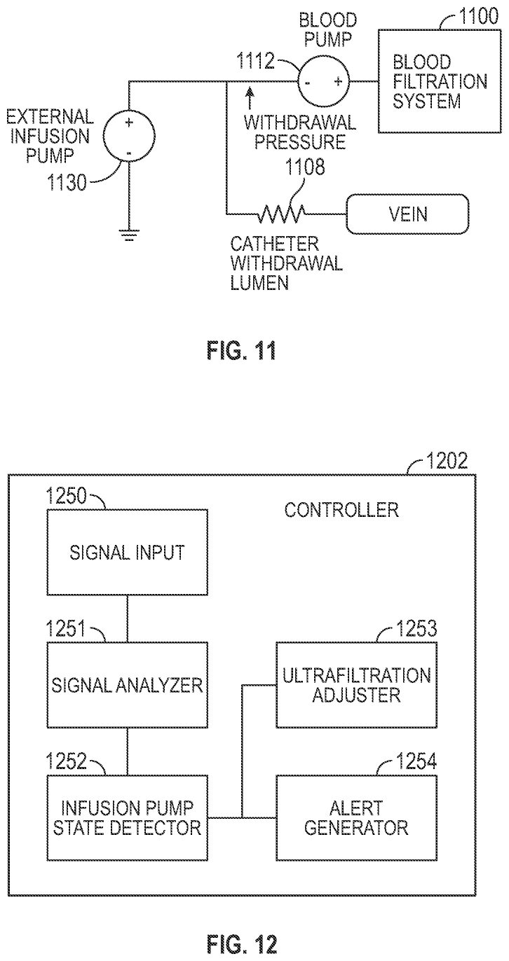

FIG. 11 is a circuit diagram illustrating an electrical circuit model for the pneumatic connections of an external infusion pump 1130, a blood pump 1112, and a catheter 1108, an a blood filtration system 1100, analogous to external infusion pump 130, blood pump 112, catheter 108, and blood filtration system 100 as in the setup illustrated in FIG. 1. It is noted that through shown separately in FIG. 11, blood pump 1112 is part of blood filtration system 1100. It is also noted that detection of the operational state of external infusion pump 130 using the withdrawal pressure signal is discussed as an example with reference to FIG. 11, the technique also applies when the infusion pressure signal is used. In FIG. 11, catheter 1108 is represented by a resistor, with its resistance representing the fluid flow resistance of the withdrawal catheter lumen, and the withdrawal pressure can be represented by a voltage.

This detection method applies are both external infusion pump 1130 and blood pump 1112 are turned on. Periodically, blood pump 112 is turned off for a brief period. During this brief period when blood pump 112 is in an off-state, no blood can move into the filter of blood filtration system 1100 because the blood is blocked by the rollers on the peristaltic pump raceway of blood pump 112, and the infusion fluid can travel back into the patient's vein through the withdrawal lumen of catheter 1108. The fluid flow resistance in the withdrawal lumen will cause a pressure to be developed at the pressure sensor that senses the withdrawal pressure signal. This step function change in the withdrawal pressure signal can be detected in the time domain automatically (e.g., using pattern recognition) or manually (e.g., by observing the change in the withdrawal pressure signal. If the step function is detected, the external infusion pump 1130 is in the on-state. If the step function is not detected, the external infusion pump 1130 is in the off-state.

In various embodiments, the change in pressure signal due to blood pump pause, as discussed above with reference to FIG. 11, can be used to detect the on/off state of external infusion pump 130 alone or in combination with the Fourier Transform and/or the autocorrelation. For example, when better reliability (e.g., higher sensitivity to changes in the detection of the operational state of external infusion pump 130) is desired, detection based on the change in pressure signal due to blood pump pause can be used as another orthogonal approach to complement the detection based on the Fourier Transform and/or the autocorrelation. In one embodiment, a pressure signal is sensed using pressure sensor 124A or 124B, and the operational state of external infusion pump 130 is detected using the Fourier Transform of the pressure signal, the autocorrelation of the pressure signal, and the change in the pressure signal due to blood pump pause. In another embodiment, two pressure signals are sensed using pressure sensors 124A and 124B, and the operational state of external infusion pump 130 is detected using the Fourier Transform of each of the two pressure signals, the autocorrelation of each of the two pressure signals, and the change in each of the two pressure signals due to blood pump pause.

### Detection Using Hematocrit Signal(s)

The one or more hematocrit signals sensed by hematocrit sensor 140A and/or hematocrit sensor 140B can be used to detect the operational state of external infusion pump 130. Hematocrit sensor 140A and/or hematocrit sensor 140B can each be an optical sensor providing for continuous hematocrit monitoring in withdrawal line 104 and/or infusion line 106 of blood filtration system 100. When external infusion pump 130 is in the on-state, the blood in blood circuit 103 is diluted by the infusion fluid, and hence the hematocrit decreases. When external infusion pump 130 is in the off-state, the blood in blood circuit 103 is not diluted by the infusion fluid, and hence the hematocrit increases. Thus, the on/off state of external infusion pump 130 can be detected by comparing each sensed hematocrit signal to a threshold (e.g., a threshold established based on know values of hematocrit of the blood when external infusion pump 130 is turned on and off).

In various embodiments, the hematocrit-based detection of the on/off state of external infusion pump 130 can be used alone or in combination of the pressure-based detection. For example, when better reliability (e.g., higher sensitivity to changes in the detection of the operational state of external infusion pump 130) is desired, detection using the hematocrit signal(s) can be applied to complement the detection based on the pressure signal(s).

### Controller Example

FIG. 12 is a block diagram illustrating an embodiment of a controller 1202, which can represent an example of controller 102. Controller 102 can perform the detection of the operational state of external infusion pump 130 using the pressure signal(s) and/or the detection using the hematocrit signal(s) as discussed above. Controller 102 can optionally further perform automatic adjustment of operation of blood filtration system 100 and/or alert generation using an outcome of the detection.

Controller 1202 can include a signal input 1250, a signal analyzer 1251, and an infusion pump state detector 1252. Signal input 1250 can receive one or more signals used for detecting the operational state of external infusion pump 130. In various embodiments, the one or more signals can include one or more of the withdrawal pressure signal sensed using withdrawal pressure sensor 124A, the infusion pressure signal sensed using infusion pressure sensor 124B, and the hematocrit signal sensed using hematocrit sensor 140A or 140B.

Signal analyzer 1251 can analyze the one or more signals received by signal input 1250. The analysis can include, but are not limited to, any one or any combination of the examples I-III:
I. processing the withdrawal pressure signal to produce:
   (a) a Fourier transform of the withdrawal pressure signal;
   (b) an autocorrelation of the withdrawal pressure signal; and/or
   (c) a change in the withdrawal pressure signal associated with a pause of blood pump 112;
II. processing the infusion pressure signal to produce:
   (a) a Fourier transform of the infusion pressure signal;
   (b) an autocorrelation of the infusion pressure signal; and/or
   (c) a change in the infusion pressure signal associated with a pause of blood pump 112; and
III. processing the hematocrit signal and produce a change in the hematocrit resulting from change in the operational state of external infusion pump 130.

Infusion pump state detector 1252 can detect the operational state of external infusion pump 130 using results of the analysis of the one or more signals performed by signal analyzer 1251. In various embodiments, infusion pump state detector 1252 can detect the operational state of external infusion pump 130 using results of the analysis including any one or any combination of analyses I(a), I(b), I(c), II(a), II(b), II(c), and III as discussed above regarding signal analyzer 1251. The selection of the one or more signals and analyses used in the detection of the operational state of external infusion pump 130 can be based on existing sensors in each instance (e.g., a specific product model) of blood filtration system 100, required or desired level of reliability, and/or the like. Examples of combinations of analyses that can be used for detecting the operational state of external infusion pump 130 include:
I(a), I(b), I(c), II(a), II(b), II(c), and III;
I(a), I(b), I(c), II(a), II(b), and II(c);
I(a), I(b), II(a), II(b), and III;
I(a), I(b), II(a), and II(b);
I(a), I(c), II(a), II(c), and III;
I(a), I(c), II(a), and II(c)
I(b), I(c), II(b), II(c), and III;
I(b), I(c), II(b), and II(c)
I(a), II(a), and III;
I(a) and II(a);
I(b), II(b), and III;
I(b) and II(b);
I(c), II(c), and III;
I(c) and II(c);
I(a), I(b), I(c), and III;
I(a), I(b), and I(c);
I(a), I(b), and III;
I(a) and I(b);
I(a), I(c), and III;
I(a) and I(c);
I(b), I(c), and III;
I(b) and I(c);
I(a) and III;
I(b) and III;
I(c) and III;
II(a), II(b), II(c), and III;
II(a), II(b), and II(c);
II(a), II(b), and III;
II(a) and II(b);
II(a), II(c), and III;
II(a) and II(c);
II(b), II(c), and III;
II(b) and II(c);
II(a) and III;
II(b) and III; and
II(c) and III.

Controller 1202 can optionally further include an ultrafiltration adjuster 1253 and/or an alert generator 1254, to provide blood filtration system 100 with one or more automatic responses to changes in the detected operational state of external infusion pump 130. Ultrafiltration adjuster 1253 can adjust the UR of filtration pump 116 based on the detected operational state of external infusion pump 130 (e.g., to maintain a desirable net fluid removal rate). In various embodiments, ultrafiltration adjuster 1253 turns filtration pump 116 (and blood pump 112) off in response to the detection that external infusion pump 130 is in the off-state (e.g., until the user takes action).

The Fourier Transform of the pressure signal also indicates the infusion rate. In various embodiments, infusion pump state detector 1252 can also determine a magnitude of flow rate change of external infusion pump 130 (e.g., change in the infusion rate) using the Fourier Transform as the operational state of external infusion pump 130 (e.g., in addition to, or instead of, the on/off-state). For example, as illustrated in FIGS. 3A and 3B, the frequency of the spike in the FFT of the pressure signal created by external infusion pump 130 is indicative of the infusion rate. This frequency increases when the infusion rate increases and decreases when the infusion rate decreases. Thus, signal analyzer 1251 can process the withdrawal pressure signal or the infusion pressure signal to produce the Fourier Transform of the pressure signal, and infusion pump state detector 1252 can determine the change of the infusion rate using the Fourier Transform. Ultrafiltration adjuster 1253 can then adjust the UR of filtration pump 116 to account for the change of the infusion rate as determined by infusion pump state detector 1252 to maintain fluid balance.

Alert generator 1254 can generate an alert signal indicative of a change in the detected state of external infusion pump 130. In various embodiments, the change in the detected state of external infusion pump 130 (e.g., from the on-state to the off-state) can result in an automatic response by ultrafiltration adjuster 1253 and/or in alert signal to inform the user (e.g., to take manual actions when necessary).

In various embodiments, controller 1202 can operate under an initial learning mode and a therapy mode. Under the initial learning mode, controller 1202 can learn characteristics of the one or more signals received by signal input 1250 (e.g., through machine-learning) during each state of operational states of external infusion pump 130. The operational states includes at least an on-state during which external infusion pump 130 pumps the infusion fluid and an off-state during which external infusion pump 130 does not pump the infusion. This allows infusion pump state detector 1252 to detect the operational state of external infusion pump 130 using the learned characteristics of the one or more signals during each state. Under the therapy mode (e.g., when blood filtration system 100 and external infusion pump 130 have been turned on), controller 1202 can detect the operational state of external infusion pump 130 continuously or intermittently (e.g., periodically). For example, controller 1202 can detect the operational state of external infusion pump 130 periodically at a period between 5 minutes and 30 minutes, with 10 minutes being a specific example.

In various embodiments, controller 1202 can operate under the initial learning mode to learn the characteristics of the one or more signals received by signal input 1250 associated with different types and/or models of external infusion pump 130 (e.g., through machine-learning). In practice, each instance of blood filtration system 100 as a product can be used with multiple instances of external infusion pump 130 as different products provided by one or more manufacturers and having different characteristics. Controller 1202 can store the learned characteristics associated with each different instance of external infusion pump 130 in its memory, and retrieve the stored information based on which specific external infusion pump 130 is coupled to blood filtration system 100 each time when they are used to treat a patient. In this manner, controller 1202 need to learn the characteristics of the one or more signals received by signal input 1250 associated with each different type or model of external infusion pump 130 only once.

In various embodiments, settings of blood filtration system 100 need to be adjusted based on whether external infusion pump 130 is directly coupled to withdrawal line 104 or infusion line 106, such as when blood filtration system 100 is a continuous veno-venous hemofiltration (CVVH) system. Infusion pump state detector 1252 can determine the location of external infusion pump 130 (e.g., whether it is directly coupled to withdrawal line 104 or infusion line 106) as the operational state (e.g., in addition to the on/off-state and/or the infusion rate) using the withdrawal pressure signal sensed by withdrawal pressure sensor 124A and the infusion pressure signal sensed by infusion pressure sensor 124B. It is noted that in the context related to the location of external infusion pump 130, “directly coupled to withdrawal line 104” means an access port (e.g., 120A or 120B) on withdrawal line 104 is used as the infusion port, and “directly coupled to infusion line 106” means an access port (e.g., 120C) on infusion line 106 is used as the infusion port. For example, infusion pump state detector 1252 can determine the location of external infusion pump 130 using any one or any combination of the following approaches:
(1) The withdrawal pressure signal is sensed by withdrawal pressure sensor 124A, and the infusion pressure signal is sensed by infusion pressure sensor 124B. Blood pump 112 is turned off periodically while external infusion pump 130 remains on. Infusion pump state detector 1252 determines the location of external infusion pump 130 by detecting a sudden increase in each of the withdrawal pressure and the infusion pressure. If the sudden increase is detected from the withdrawal pressure, external infusion pump 130 is directly coupled to withdrawal line 104. If the sudden increase is detected from the infusion pressure, external infusion pump 130 is directly coupled to infusion line 104.
(2) External infusion pump 130 is turned on before the blood pump 112 is turned on (e.g., at the beginning of a therapy session). Infusion pump state detector 1252 receives the withdrawal pressure signal and the infusion pressure signal sensed when external infusion pump 130 is on but blood pump 112 is off and determines the location of external infusion pump 130 by comparing the withdrawal pressure with the infusion pressure. If the withdrawal pressure is higher than the infusion pressure, external infusion pump 130 is directly coupled to withdrawal line 104. If the withdrawal pressure is lower than the infusion pressure, external infusion pump 130 is directly coupled to infusion line 104.
(3) The withdrawal pressure signal is sensed by withdrawal pressure sensor 124A, and the infusion pressure signal is sensed by infusion pressure sensor 124B. Frequency analysis of each of the withdrawal pressure signal and the infusion pressure signal is performed by signal analyzer 1251 to provide the frequency content of each pressure signal. Infusion pump state detector 1252 determines the location of external infusion pump 130 by detecting a characteristic frequency of external infusion pump 130 from the frequency content of each of the withdrawal pressure signal and the infusion pressure signal. If the characteristic frequency is detected from the withdrawal pressure, external infusion pump 130 is directly coupled to withdrawal line 104. If characteristic frequency is detected from the infusion pressure, external infusion pump 130 is directly coupled to infusion line 104.
(4) The withdrawal pressure signal is sensed by withdrawal pressure sensor 124A, and the infusion pressure signal is sensed by infusion pressure sensor 124B. Blood pump 112 is turned off periodically while external infusion pump 130 remains on. Autocorrelation of each of the withdrawal pressure signal and the infusion pressure signal recorded while blood pump 112 is off is calculated by signal analyzer 1251. Infusion pump state detector 1252 determines the location of external infusion pump 130 by differentiate between a periodic signal and random noise based on the autocorrelation of each pressure signal. If a periodic signal is indicated by the autocorrelation of the withdrawal pressure, external infusion pump 130 is directly coupled to withdrawal line 104. If a periodic signal is indicated by the autocorrelation of the infusion pressure, external infusion pump 130 is directly coupled to infusion line 104.
   In various embodiments, controller 1202 can notify the user of the location of external infusion pump 130 and/or control the operation of blood filtration system 100 according to the location of external infusion pump 130, among other things. When external infusion pump 130 is connected to blood filtration system 100 by the user, controller 1202 can verify the location and notify the user of the outcome of the verification before the user starts the therapy.

### Detection Method Examples

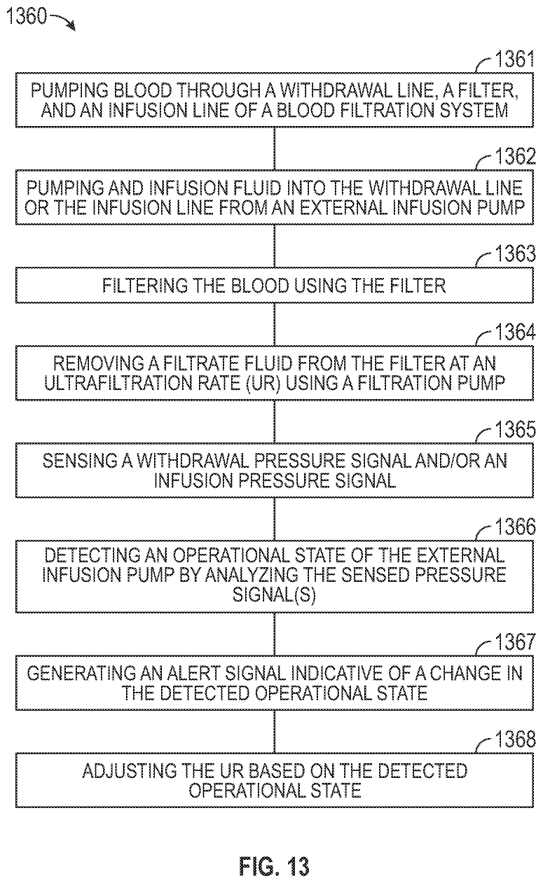

FIG. 13 is a flowchart illustrating an embodiment of a method 1360 for operating a blood filtration system coupled to an external infusion pump. The blood filtration system is coupled to a patient through a catheter to reduce one or more plasma constituents in blood of the patient and is coupled to the external infusion pump for infusing an infusion fluid to the patient through the blood filtration system. In one embodiment, method 1360 is performed with blood filtration system 100 and external infusion pump 130. Controller 102 (or 1202) can allow blood filtration system 100 to perform method 1360.

At 1361, the blood is pumped through a withdrawal line coupled to the catheter, a filter coupled to the withdrawal line, and an infusion line coupled between the filter and the catheter at an extracorporeal blood flow rate (EBFR) using a blood pump engaged with the withdrawal line. The withdrawal line, the filter, the infusion line (also referred to as the return line), and the blood pump are among the components of the blood filtration system.

At 1362, the infusion fluid is pumped into the withdrawal line or the infusion line from the external infusion pump. The infusion fluid (also referred to as replacement fluid) can include saline and one or more diagnostic and/or therapeutic substances.

At 1363, the blood is filtered using the filter. The filtering removes portions of the one or more plasma constituents in the blood.

At 1364, a filtrate fluid including the removed portions of the one or more plasma constituents is removed from the filter at an ultrafiltration rate (UR) using a filtration pump. The filtration pump is a component of the blood filtration system.

At. 1365, one or more signals indicative of an operational state of the external infusion pump are sensed. The one or more signals include at least a withdrawal pressure signal indicative of a pressure in the withdrawal line and/or an infusion pressure signal indicative of a pressure in the infusion line. In some embodiments, the one or more signals includes a hematocrit signal indicative of a hematocrit of the blood in the withdrawal line and/or a hematocrit signal indicative of a hematocrit of the blood in the infusion line.

At 1366, the operational state of the external infusion pump is detected by analyzing the one or more signals sensed at 1365. A controller of the blood filtration system can perform the functions of controller 1202 related to the detection, as discussed above with reference to FIG. 12. In various embodiments, the detection is based on analysis of at least one of the withdrawal pressure signal or the infusion pressure signal. In various embodiments, the detection is based on analysis of one of the hematocrit signal in addition to the withdrawal pressure signal and/or the infusion pressure signal for higher detection sensitivity and reliability. In various embodiments, the operational state of the external infusion pump to be detection can include an on/off-state of the external infusion pump, an infusion rate of the external infusion pump, and/or a location of the external infusion pump (e.g., whether the external infusion pump is directly coupled to the withdrawal line or directly coupled to the infusion line).

At 1367, an alert signal indicative of a change in the detected operational state of the external infusion pump is generated. The alert signal informs a user of a potential need for adjusting the blood filtration system, adjusting the external infusion pump, and/or attending the patient.

At 1368, the ultrafiltration rate (UR) of the filtration pump is adjusted based on the detected operational state of the external infusion pump. In various embodiments, the controller of the blood filtration system can automatically adjust the UR in response to the change in the detected operational state of the external infusion pump or to allow the user to adjust the UR manually. In one embodiment, the detected operational state of the external infusion pump includes a magnitude of flow rate change of the external infusion pump, and the UR is adjusted based on the determined magnitude of the flow rate change of the external infusion pump.

In various embodiments, a controller of the blood filtration system, such as controller 102 or 1202 of blood filtration system as discussed above, can control the operation of the blood filtration system for performing method 1360 without direction communication (e.g., via a wired or wireless communication link coupled directly between the blood filtration system and the external infusion pump).

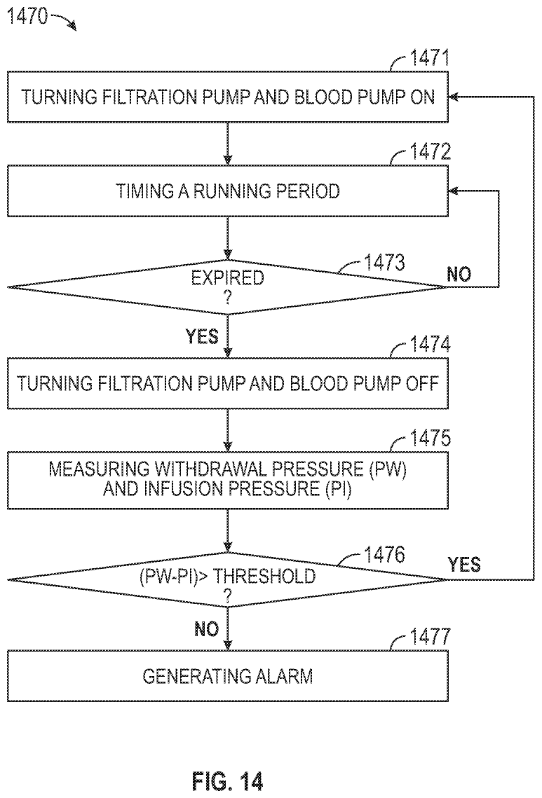

FIG. 14 is a flowchart illustrating an embodiment of a method 1470 for operating a blood filtration system while monitoring on-off state of an external infusion pump periodically. This embodiment can represent an alternative approach to I(c) and II(c) of the analysis performed by signal analyzer 1251 as discussed above (I(c): processing the withdrawal pressure signal to produce a change in the withdrawal pressure signal associated with a pause of blood pump 112; II(c): processing the infusion pressure signal to produce a change in the infusion pressure signal associated with a pause of blood pump 112).

The blood filtration system is coupled to a patient through a catheter to reduce one or more plasma constituents in blood of the patient and is coupled to the external infusion pump for infusing an infusion fluid to the patient through the blood filtration system. In one embodiment, method 1470 is performed with blood filtration system 100 and external infusion pump 130. Controller 102 (or 1202) can allow blood filtration system 100 to perform method 1470.

At 1471, the filtration pump and the blood pump of the blood filtration system are turned on. At 1472, a running period is timed. The running period starts or restarts every time when the filtration pump and the blood pump are turned on. In various embodiments, the running period can be between 5 minutes and 30 minutes, with 10 minutes being a specific example. If the running period expires at 1473, the filtration pump and the blood pump are turned off at 1474. At 1475, a withdrawal pressure (PW) is measured from a withdrawal pressure signal sensed from a withdrawal line of the blood filtration system, and an infusion pressure (PI) is measured from an infusion pressure signal sensed from an infusion line (also referred to as a return line) of the blood filtration system. If the difference between the withdrawal pressure and the infusion pressure (PW−PI) is greater than a threshold at 1476, the filtration pump and the blood pump of the blood filtration system are turned (back) on at 1471. If the difference between the withdrawal pressure and the infusion pressure (PW−PI) is not greater than a threshold at 1476, an alarm is generated at 1477. The filtration pump and the blood pump remain off until a user takes action in response to the alarm.

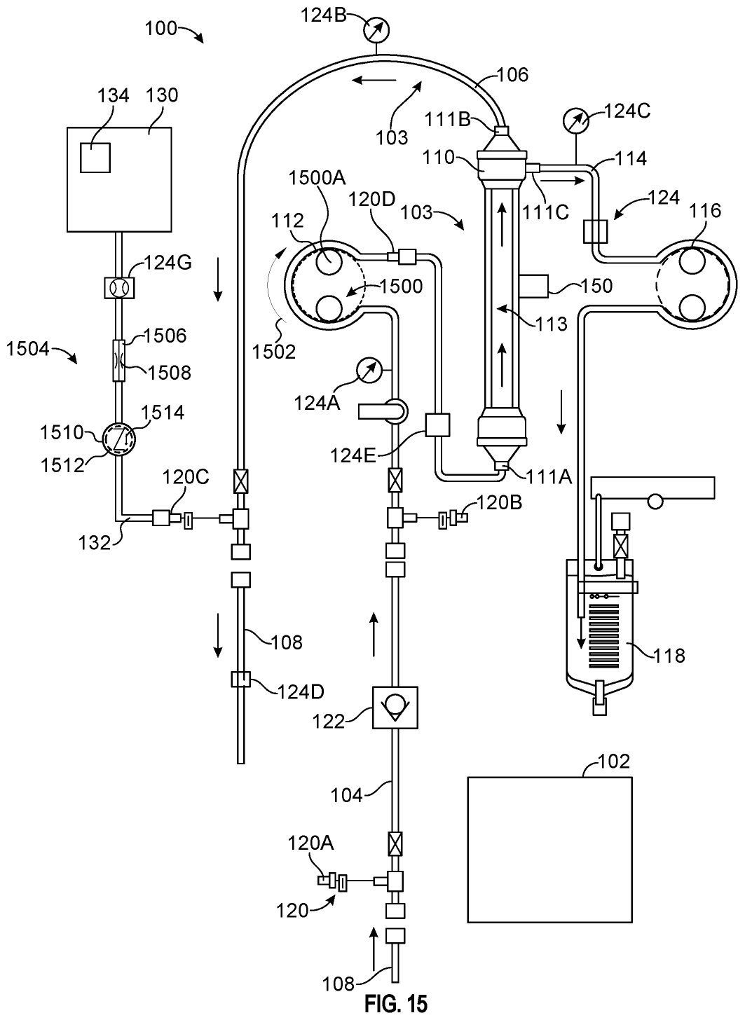

FIG. 15 is an illustration of another embodiment of the blood filtration system 100 coupled to the external infusion pump 130. The blood filtration system 100 can include the blood circuit 103. The external infusion pump can provide a fluid to the blood circuit 103. For instance, the external infusion pump 130 can pump fluid through the external infusion line 132 and into the access port 120C (e.g., an infusion port, or the like).

The blood filtration system 100 can include the one or more sensors 124, for example a flow sensor 124G. The flow sensor 124G can be in fluidic communication with one or more of the external infusion line 130 or the blood circuit 103. The flow sensor 124G can sense a flow rate signal indicative of flow rate within one or more of the external infusion line 130 or the blood circuit 103.

In some approaches, operation of the blood pump 112 (e.g., a circuit pump, or the like) or the external infusion pump 130 can generate variations in flow of fluid in the external infusion line 132 or the blood circuit 103. For example, the blood pump 112 can include a peristaltic pump. The peristaltic pump 112 can include one or more rollers 1500 that engage with the blood circuit 103 to pump fluid in the blood circuit 103. For example, the rollers 1500 can move relative to the blood circuit 103. The rollers 1500 can compress (e.g., squeeze, constrict, pinch, smush, draw, grip, or the like) the withdrawal line 104 to pump fluid in the blood circuit 103. At least one of the rollers 1500 can disengage from the blood circuit 103 during operation of the blood pump 112. For example, a first roller 1500A can be engaged with the blood circuit 103 for a first half of a cycle of the blood pump 112. The first roller 1500A can be disengaged for a second half of the cycle of the blood pump 112. FIG. 15 shows arrow 1502 indicating direction of flow through the blood circuit 103 during operation of the blood pump 112. The blood pump 112 can pump blood toward the filter 110.

The engagement (and disengagement) of the rollers 1500 with the blood circuit 103 can generate variations in flow of fluid in the blood circuit 103 (or the external infusion line 132 in fluidic communication with the blood circuit 103). For instance, the engagement of the rollers 1500 with the blood circuit 103 can temporarily reverse flow (e.g., opposite the direction of the arrow 1502) of fluid in the blood circuit 103. The temporary reversal in flow can occur in correspondence with movement of the rollers 1500. For instance, the roller 1500A can temporarily reverse flow when the roller 1500A initially engages with the blood circuit 103. The roller 1500A can continue to move relative to the blood circuit 103 and fully engage with the blood circuit 103 (pumping fluid in the direction of the arrow 1502). In another example, flow is temporarily reversed when the rollers 1500 disengage from the blood circuit. For instance, flow can temporarily be reversed in correspondence with the compressed blood circuit 103 (e.g., tubing, or the like) returns to an uncompressed state.

The blood circuit 103 can communicate fluid with the external infusion line 103. Accordingly, the variations in flow of fluid in the blood circuit 103 can correspondingly vary flow of fluid in the external infusion line 132. Variations in flow of fluid in the external infusion line 132 can affect the flow rate signal sensed by the flow sensor 124G. For instance, the reversal in flow of fluid in the blood circuit 103 can correspondingly reverse flow in the external infusion line 132. The (temporary) reversal in flow in the external infusion line 132 can induce noise in the flow rate signal sensed by the flow sensor 124G. Noise can decrease accuracy (or precision) of the correspondence between the flow rate signal and the flow rate of fluid.

The blood filtration system 100 can include a damping system 1504. The damping system 1504 can damp flow of fluid. The damping system 1504 can damp flow (or variations in flow) of fluid in one or more of the external infusion line 132 or the blood circuit 103. The damping system 1504 can cooperate with the flow sensor 124G to damp variations in the flow rate signal based on operation of one or more of the external infusion pump 130 or the blood pump 112 (e.g., a circuit pump. or the like). In an example, damping flow of fluid in one or more of the external infusion line 132 or the blood circuit 103 can minimize noise induced in the flow rate signal sensed by the flow sensor 124G. For instance, the damping system 1504 can damp the temporary reversal in flow in the external infusion line 132 (or the blood circuit 103). Damping the temporary reversal in flow can minimize noise in the flow rate signal. Accordingly, the damping system 1504 can enhance the accuracy (or precision) of the correspondence between the flow rate signal and the flow rate of the fluid.

The damping system 1504 can include a flow restrictor 1506. The flow restrictor 1506 can have an orifice 1508 to restrict flow of fluid. For instance, the flow restrictor 1506 can be in fluidic communication with one or more of the external infusion line 132, the blood circuit 103. or the flow sensor 124G. The orifice 1508 can reduce cross-sectional area of one or more of the external infusion line 132 or the blood circuit 103. Accordingly, the flow restrictor 1506 having the orifice 1508 can restrict flow of fluid in one or more of the external infusion line 132 or the blood circuit 103.

The damping system 1504 can include a check-valve 1510. The check-valve 1510 can be in fluidic communication with one or more of the external infusion line 132, the blood circuit 103, the flow sensor 124G, or the flow restrictor 1506. The check-valve 1510 can have an open configuration and a closed configuration. In the open configuration, the check-valve 1510 can permit flow of fluid through a valve body 1512 in a first direction (shown in FIG. 15 with arrow 1514). In the closed configuration, the check-valve 1510 can inhibit the flow of fluid through the valve body 1512 in a second direction (e.g., opposite the arrow 1514, or the like). The check-valve 1510 can inhibit flow in the second direction as the check-valve 1510 transitions between the open configuration to the closed configuration. Accordingly, the check-valve 1510 can partially inhibit flow in the second direction, or the check-valve 1510 can fully inhibit flow in the second direction. In an example, the check-valve 1510 can inhibit the temporary reversal in flow of fluid in the external infusion line 132 or the blood circuit 103.

The check-valve 1510 can cooperate with the flow restrictor to damp flow through the external infusion line 132 or the blood circuit 103. For instance, the check-valve can inhibit flow in the second direction in correspondence with the temporary reversal of flow in one or more of the external infusion line 132 or the blood circuit 103. The flow restrictor 1506 can minimize a magnitude of the reversal of flow. For instance, the flow of fluid through the orifice 1508 can damp the reversal of flow in correspondence with the check valve 1510 in the open configuration (or in correspondence with the check valve transitioning between the open configuration to the closed configuration). Accordingly, the damping system can enhance the accuracy (or precision) of the correspondence between the flow rate signal sensed by the flow sensor 124G and the flow rate of the fluid.

In another example, the damping system 1504 can be located between the external infusion pump 130 and the access port 120C. The damping system 1504 can be located between the external infusion pump 130 and the access port 120C. For instance, FIG. 15 shows the flow sensor 124G adjacent the external infusion pump 130. Fluid pumped by the external infusion pump 130 can flow through the external infusion line 132 to the flow sensor 124G. FIG. 15 shows fluid can flow from the flow sensor 124G to the flow restrictor 1506. In another example, FIG. 15 shows fluid can flow from the flow restrictor 1506 to the check-valve 1510. In yet another example, fluid can flow from the flow sensor 124G to the check-valve 1510. Fluid can flow from the check-valve 1510 to the flow restrictor 1506. Accordingly, fluid pumped by the external infusion pump 130 can flow through the check-valve 1510 after flowing through the 1506, or can flow through the flow restrictor 1506 after flowing through the check-valve 1510.

The blood filtration system 100 can use the flow rate sensor 124G to determine an amount of fluid pumped into the blood circuit 103 by the external infusion pump 130. The damping system 100 can enhance the determination of the amount of fluid pumped into the blood circuit 103 by the external infusion pump 130. For instance, the damping system 1504 can enhance the accuracy (or precision) of the correspondence between the flow rate signal sensed by the flow sensor 124G and the flow rate of the fluid. The flow rate of the fluid is associated with the amount fluid pumped into the blood circuit 103. Accordingly, enhanced accuracy (or precision) in the flow rate signal can enhance the accuracy (or precision) for determining the amount of fluid pumped into the blood circuit 103 by the external infusion pump 130. In another example, enhancing the accuracy (or precision) of the amount of fluid pumped into the blood circuit 103 can enhance operation of the blood filtration system 100. For instance, the system 100 can use the amount of fluid pumped into the blood circuit 103 by the external infusion pump 130 to adjust an ultrafiltration rate of the blood filtration system 100. Thus, enhancing the accuracy (or precision) of the flow rate signal can correspondingly enhance the accuracy (or precision) of the amount of blood constituents removed from blood of a patient using the system 100.

### Conclusion

Some non-limiting examples (Examples 1-52) of the present subject matter are provided as follows:

Example 1 is a blood filtration system configured for coupling with an external infusion pump, and to reduce one or more plasma constituents in blood of a patient, the system comprising: a blood circuit configured for coupling with a catheter, and the blood circuit is configured for fluidic communication with vasculature of a patient, wherein the blood circuit is configured to transport blood through a filter, and the filter is configured to remove one or more plasma constituents in the blood; an infusion port in fluidic communication with the blood circuit, and the infusion port configured for fluidic communication with the external infusion pump to receive an infusion fluid from the external infusion pump; at least one pressure sensor configured to sense one or more pressure signals indicative of a pressure in a portion of the blood circuit; and a blood filtration controller including a signal analyzer and an infusion pump state detector, the signal analyzer configured to analyze the one or more pressure signals, and the infusion pump state detector configured to detect an operational state of the external infusion pump using an outcome of the analysis.

In Example 2, the subject matter of Example 1 optionally includes a hematocrit sensor configured for coupling with the blood circuit to sense a hematocrit signal indicative of a hematocrit of the blood; and wherein the signal analyzer is configured to analyze the hematocrit signal and the one or more pressure signals to detect the operational state of the infusion pump.

In Example 3, the subject matter of any one or more of Examples 1-2 optionally include wherein the signal analyzer is configured to analyze the one or more pressure signals by processing an individual pressure signal of the one or more pressure signals to produce at least one of a Fourier transform of the individual pressure signal, an autocorrelation of the individual pressure signal, or a change in the individual pressure signal associated with a pause of the blood pump.

In Example 4, the subject matter of any one or more of Examples 1-3 optionally include wherein the blood filtration controller includes a pump adjuster configured to adjust an ultrafiltration rate based on the detected operational state of the external infusion pump.

In Example 5, the subject matter of any one or more of Examples 1-4 optionally include wherein: the blood circuit includes a withdrawal line configured for coupling with the catheter to receive the blood from the patient through the catheter; the blood circuit includes an infusion line configured for coupling with the catheter to return blood into the patient through the catheter; and the blood circuit includes the filter, and the filter is configured for coupling between the withdrawal line and the infusion line, and the filter is configured to produce filtered blood by removing portions of the one or more plasma constituents in the blood.

In Example 6, the subject matter of Example 5 optionally includes wherein: the at least one pressure sensor includes one or more of a withdrawal pressure sensor or an infusion pressure sensor; the withdrawal pressure sensor is configured for coupling with the withdrawal line to sense a withdrawal pressure signal indicative of a pressure in the withdrawal line; the infusion pressure sensor is configured for coupling with the infusion line to sense an infusion pressure signal indicative of a pressure in the infusion line; and the one or more pressure signals includes at least one of the withdrawal pressure signal or the infusion pressure signal.

In Example 7, the subject matter of any one or more of Examples 5-6 optionally include a blood pump configured to engage the withdrawal line to pump the blood through the withdrawal line and into the filter at an extracorporeal blood flow rate; and a filtration pump configured to remove a filtrate fluid including the removed portions of the one or more plasma constituents from the filter at an ultrafiltration rate.

In Example 8, the subject matter of any one or more of Examples 1-7 optionally include a damping system, the damping system is configured to damp flow of fluid, the damping system including: an external infusion line configured for fluidic communication with the infusion port; a flow restrictor in fluidic communication with the external infusion line, the flow restrictor having an orifice to restrict flow through the external infusion line; and a check-valve in fluidic communication with one or more of the flow restrictor or the external infusion line, wherein: the check-valve is configured to permit flow of fluid through a valve body in a first direction; and the check-valve is configured to restrict the flow of fluid through the valve body in a second direction.

In Example 9, the subject matter of Example 8 optionally includes a blood circuit pump in fluidic communication with the infusion port, and the circuit pump is configured to pump fluid in one or more of the external infusion line or the blood circuit.

In Example 10, the subject matter of Example 9 optionally includes wherein operation of one or more of the circuit pump or the external infusion pump generates variations in flow of fluid in one or more of the external infusion line or the blood circuit, and the damping system is configured to damp the variations in flow of fluid.

In Example 11, the subject matter of any one or more of Examples 9-10 optionally include wherein the pump is a peristaltic pump.

In Example 12, the subject matter of any one or more of Examples 9-11 optionally include a flow sensor in communication with one or more of the external infusion line or the blood circuit, and the flow sensor is configured to sense a flow rate signal indicative of flow rate within one or more of the external infusion line or the blood circuit.

In Example 13, the subject matter of Example 12 optionally includes wherein the damping system cooperates with the flow sensor to damp variations in the flow rate signal based on operation of one or more of the external infusion pump or the circuit pump.

In Example 14, the subject matter of any one or more of Examples 12-13 optionally include wherein the damping system is located between the flow sensor and the infusion port.

Example 15 is a blood filtration system configured for coupling with an external infusion pump, and to reduce one or more plasma constituents in blood of a patient, the system comprising: a withdrawal line configured for coupling with a catheter to receive the blood from the patient through the catheter; an infusion line configured for coupling with the catheter to return filtered blood into the patient through the catheter; an infusion port in fluidic communication with the withdrawal line or the infusion line and configured to be coupled to the external infusion pump to receive an infusion fluid from the external infusion pump; a filter configured for coupling between the withdrawal line and the infusion line and to produce the filtered blood by removing portions of the one or more plasma constituents in the blood; a blood pump configured to engage the withdrawal line to pump the blood through the withdrawal line and into the filter at an extracorporeal blood flow rate (EBFR); a filtration pump configured to remove a filtrate fluid including the removed portions of the one or more plasma constituents from the filter at an ultrafiltration rate (UR); at least one of a withdrawal pressure sensor and an infusion pressure sensor, the withdrawal pressure sensor configured to be coupled to the withdrawal line to sense a withdrawal pressure signal indicative of a pressure in the withdrawal line, the infusion pressure sensor configured to be coupled to the infusion line to sense an infusion pressure signal indicative of a pressure in the infusion line; and a controller including a signal analyzer and an infusion pump state detector, the signal analyzer configured to analyze at least one of the withdrawal pressure signal or the infusion pressure signal, the infusion pump state detector configured to detect an operational state of the external infusion pump using an outcome of the analysis.

In Example 16, the subject matter of Example 15 optionally includes a hematocrit sensor configured to be coupled to at least one of the withdrawal line or the infusion line to sense a hematocrit signal indicative of a hematocrit of the blood, and wherein the signal analyzer is configured to analyze the hematocrit signal and the at least one of the withdrawal pressure signal or the infusion pressure signal.

In Example 17, the subject matter of Example 16 optionally includes wherein the signal analyzer is configured to analyze the hematocrit signal, the withdrawal pressure signal, and the infusion pressure signal.

In Example 18, the subject matter of any one or more of Examples 15-17 optionally include wherein the signal analyzer is configured to analyze the at least one of the withdrawal pressure signal or the infusion pressure signal by processing each pressure signal of the at least one of the withdrawal pressure signal or the infusion pressure signal to produce at least one of a Fourier transform of the each pressure signal, an autocorrelation of the each pressure signal, or a change in the each pressure signal associated with a pause of the blood pump.

In Example 19, the subject matter of Example 18 optionally includes the withdrawal pressure sensor and the infusion pressure sensor, and wherein the signal analyzer is configured to analyze the withdrawal pressure signal and the infusion pressure signal.

In Example 20, the subject matter of Example 19 optionally includes a hematocrit sensor configured to be coupled to at least one of the withdrawal line or the infusion line to sense a hematocrit signal indicative of a hematocrit of the blood, and wherein the signal analyzer is configured to analyze the withdrawal pressure signal, the infusion pressure signal, and the hematocrit signal.

In Example 21, the subject matter of Example 20 optionally includes wherein the signal analyzer is configured to analyze the hematocrit signal by processing the hematocrit signal to produce a change in the hematocrit resulting from change in the operational state of the infusion pump.

In Example 22, the subject matter of any one or more of Examples 18-21 optionally include wherein the signal analyzer is configured to analyze the at least one of the withdrawal pressure signal or the infusion pressure signal by processing the each pressure signal to produce at least two of the Fourier transform of the each pressure signal, the autocorrelation of the each pressure signal, or the change in the each pressure signal associated with the pause of the blood pump.

In Example 23, the subject matter of any one or more of Examples 18-22 optionally include wherein the signal analyzer is configured to analyze the at least one of the withdrawal pressure signal or the infusion pressure signal by processing the each pressure signal to produce the Fourier transform of the each pressure signal, the autocorrelation of the each pressure signal, and the change in the each pressure signal associated with the pause of the blood pump.

In Example 24, the subject matter of Example 23 optionally includes the withdrawal pressure sensor and the infusion pressure sensor, and wherein the signal analyzer is configured to analyze the withdrawal pressure signal and the infusion pressure signal.

In Example 25, the subject matter of Example 24 optionally includes a hematocrit sensor configured to be coupled to at least one of the withdrawal line or the infusion line to sense a hematocrit signal indicative of a hematocrit of the blood, and wherein the signal analyzer is configured to analyze the withdrawal pressure signal, the infusion pressure signal, and the hematocrit signal.

In Example 26, the subject matter of Example 25 optionally includes wherein the signal analyzer is configured to analyze the hematocrit signal by processing the hematocrit signal to produce a change in the hematocrit resulting from change in the operational state of the infusion pump.

In Example 27, the subject matter of any one or more of Examples 23-26 optionally include wherein the infusion pump state detector is configured to detect whether the external infusion pump is in direct fluidic communication with the withdrawal line or the infusion line via the infusion port.

In Example 28, the subject matter of any one or more of Examples 18-27 optionally include wherein the controller further comprises an ultrafiltration adjuster configured to adjust the ultrafiltration rate of the filtration pump based on the detected operational state of the external infusion pump.

In Example 29, the subject matter of Example 28 optionally includes wherein the signal analyzer is configured to produce the Fourier transform of the each pressure signal, the infusion pump state detector is configured to determine a magnitude of flow rate change of the external infusion pump using the Fourier Transform, and the ultrafiltration adjuster is configured to adjust the ultrafiltration rate of the filtration pump based on the determined magnitude of the flow rate change of the external infusion pump.

In Example 30, the subject matter of any one or more of Examples 28-29 optionally include wherein the controller further comprises an alert generator configured to generate an alert signal indicative of a change in the detected operational state of the external infusion pump.

In Example 31, the subject matter of any one or more of Examples 18-30 optionally include wherein the controller is configured to operate under an initial learning mode and a therapy mode, the signal analyzer is configured to learn characteristics of each pressure signal of the at least one of the withdrawal pressure signal or the infusion pressure signal during each state of multiple operational states of the external infusion pump under the learning mode, and the infusion pump state detector is configured to detect the operational state of the external infusion pump using the learned characteristics of the each pressure signal during the each state under the therapy mode.

In Example 32, the subject matter of Example 31 optionally includes wherein the infusion pump state detector is configured to detect the operational state of the external infusion pump periodically under the therapy mode.

In Example 33, the subject matter of any one or more of Examples 31-32 optionally include wherein the controller is configured to operate under the initial learning mode for each external infusion pump of different external infusion pumps, and the signal analyzer is configured to learn the characteristics of each pressure signal of the at least one of the withdrawal pressure signal or the infusion pressure signal during each state of multiple operational states of the each infusion pump under the learning mode and to store the learned characteristics associated with the each external infusion pump for use during the therapy mode.

In Example 34, the subject matter of any one or more of Examples 15-33 optionally include wherein the system comprises an ultrafiltration (UF) system.

In Example 35, the subject matter of any one or more of Examples 15-34 optionally include wherein the system comprises a continuous renal replacement therapy (CRRT) system.

Example 36 is a method for operating a blood filtration system coupled to a patient through a catheter to reduce one or more plasma constituents in blood of the patient and coupled to an external infusion pump for infusing an infusion fluid to the patient through the blood filtration system, the method comprising: pumping blood through a withdrawal line coupled to the catheter, a filter coupled to the withdrawal line, and an infusion line coupled between the filter and the catheter at an extracorporeal blood flow rate (EBFR) using a blood pump engaged with the withdrawal line; pumping the infusion fluid into the withdrawal line or the infusion line from the external infusion pump; removing portions of the one or more plasma constituents in the blood using the filter; removing a filtrate fluid including the removed portions of the one or more plasma constituents from the filter at an ultrafiltration rate (UR) using a filtration pump; sensing at least one of a withdrawal pressure signal indicative of a pressure in the withdrawal line or an infusion pressure signal indicative of a pressure in the infusion line; and detecting an operational state of the external infusion pump by analyzing the at least one of the withdrawal pressure signal or the infusion pressure signal.

In Example 37, the subject matter of Example 36 optionally includes sensing a hematocrit signal indicative of a hematocrit of the blood in at least one of the withdrawal line or the infusion line; and detecting the operational state of the external infusion pump by analyzing the hematocrit signal and the at least one of the withdrawal pressure signal or the infusion pressure signal.

In Example 38, the subject matter of Example 37 optionally includes wherein analyzing the hematocrit signal and the at least one of the withdrawal pressure signal or the infusion pressure signal comprises analyzing the hematocrit signal, the withdrawal pressure signal, and the infusion pressure signal.

In Example 39, the subject matter of any one or more of Examples 37-38 optionally include wherein analyzing the at least one of the withdrawal pressure signal or the infusion pressure signal comprises processing each pressure signal of the at least one of the withdrawal pressure signal or the infusion pressure signal to produce at least one of a Fourier transform of the each pressure signal, an autocorrelation of the each pressure signal, or a change in the each pressure signal associated with a pause of the blood pump.

In Example 40, the subject matter of Example 39 optionally includes wherein analyzing the at least one of the withdrawal pressure signal or the infusion pressure signal comprises analyzing the withdrawal pressure signal and the infusion pressure signal.

In Example 41, the subject matter of Example 40 optionally includes sensing a hematocrit signal indicative of a hematocrit of the blood in the withdrawal line or the infusion line; and detecting the operational state of the external infusion pump by analyzing the withdrawal pressure signal, the infusion pressure signal, and the hematocrit signal.

In Example 42, the subject matter of any one or more of Examples 39-41 optionally include wherein analyzing the at least one of the withdrawal pressure signal or the infusion pressure signal comprises analyzing the at least one of the withdrawal pressure signal or the infusion pressure signal by processing the each pressure signal to produce at least two of the Fourier transform of the each pressure signal, the autocorrelation of the each pressure signal, or the change in the each pressure signal associated with the pause of the blood pump.

In Example 43, the subject matter of Example 42 optionally includes wherein analyzing the at least one of the withdrawal pressure signal or the infusion pressure signal comprises analyzing the at least one of the withdrawal pressure signal or the infusion pressure signal by processing the each pressure signal to produce the Fourier transform of the each pressure signal, the autocorrelation of the each pressure signal, and the change in the each pressure signal associated with the pause of the blood pump.

In Example 44, the subject matter of Example 43 optionally includes wherein analyzing the at least one of the withdrawal pressure signal or the infusion pressure signal comprises analyzing the withdrawal pressure signal and the infusion pressure signal.

In Example 45, the subject matter of Example 44 optionally includes sensing a hematocrit signal indicative of a hematocrit of the blood in the withdrawal line or the infusion line, and detecting the operational state of the external infusion pump by analyzing the withdrawal pressure signal, the infusion pressure signal, and the hematocrit signal.

In Example 46, the subject matter of any one or more of Examples 44-45 optionally include determining whether the infusion fluid is pumped into the withdrawal line directly or into the infusion line directly from the external infusion pump.

In Example 47, the subject matter of any one or more of Examples 39-46 optionally include adjusting the ultrafiltration rate of the filtration pump based on the detected operational state of the external infusion pump.

In Example 48, the subject matter of Example 47 optionally includes wherein analyzing the at least one of the withdrawal pressure signal or the infusion pressure signal comprises processing the each pressure signal to produce at least the Fourier transform of the each pressure signal, and adjusting the ultrafiltration rate comprises: determining a magnitude of flow rate change of the external infusion pump using the Fourier Transform; and adjusting the ultrafiltration rate of the filtration pump based on the determined magnitude of the flow rate change of the external infusion pump.

In Example 49, the subject matter of any one or more of Examples 36-48 optionally include generating an alert signal indicative of a change in the detected operational state of the external infusion pump.

In Example 50, the subject matter of any one or more of Examples 36-49 optionally include learning characteristics of each pressure signal of the at least one of the withdrawal pressure signal or the infusion pressure signal during each state of multiple operational states of the external infusion pump under a learning mode; and detecting the operational state of the external infusion pump using the learned characteristics of the each pressure signal during the each state under a therapy mode.

In Example 51, the subject matter of Example 50 optionally includes learning the characteristics of each pressure signal of the at least one of the withdrawal pressure signal or the infusion pressure signal during each state of multiple operational states of each external infusion pump of multiple external infusion pumps under the learning mode; and detecting the operational state of the each external infusion pump using the learned characteristics of the each pressure signal during the each state of the each external infusion pump.

In Example 52, the subject matter of any one or more of Examples 50-51 optionally include detecting the operational state of the external infusion pump periodically under the therapy mode.

This application is intended to cover adaptations or variations of the present subject matter. It is to be understood that the above detailed description is intended to be illustrative, and not restrictive. Other embodiments will be apparent to those of skill in the art upon reading and understanding the above description. The scope of the present subject matter should be determined with reference to the appended claims, along with the full scope of legal equivalents to which such claims are entitled.

1. A blood filtration system configured for coupling with an external infusion pump, and to reduce one or more plasma constituents in blood of a patient, the system comprising:
a blood circuit configured for coupling with a catheter, and the blood circuit is configured for fluidic communication with vasculature of a patient, wherein the blood circuit is configured to transport blood through a filter, and the filter is configured to remove one or more plasma constituents in the blood;
an infusion port in fluidic communication with the blood circuit, and the infusion port configured for fluidic communication with the external infusion pump to receive an infusion fluid from the external infusion pump;
at least one pressure sensor configured to sense one or more pressure signals indicative of a pressure in a portion of the blood circuit;
a blood filtration controller including a signal analyzer and an infusion pump state detector, the signal analyzer configured to analyze the one or more pressure signals, and the infusion pump state detector configured to detect an operational state of the external infusion pump using an outcome of the analysis; and
wherein the blood filtration controller includes a pump adjuster configured to adjust an ultrafiltration rate based on the detected operational state of the external infusion pump.

2. The system of claim 1, further comprising:
a hematocrit sensor configured for coupling with the blood circuit to sense a hematocrit signal indicative of a hematocrit of the blood; and
wherein the signal analyzer is configured to analyze the hematocrit signal and the one or more pressure signals to detect the operational state of the infusion pump.

3. The system of claim 1, wherein the signal analyzer is configured to analyze the one or more pressure signals by processing an individual pressure signal of the one or more pressure signals to produce at least one of a Fourier transform of the individual pressure signal, an autocorrelation of the individual pressure signal, or a change in the individual pressure signal associated with a pause of the blood pump.

4. (canceled)

5. The blood filtration system of claim 1, wherein:
the blood circuit includes a withdrawal line configured for coupling with the catheter to receive the blood from the patient through the catheter;
the blood circuit includes an infusion line configured for coupling with the catheter to return blood into the patient through the catheter; and
the blood circuit includes the filter, and the filter is configured for coupling between the withdrawal line and the infusion line, and the filter is configured to produce filtered blood by removing portions of the one or more plasma constituents in the blood.

6. The blood filtration system of claim 5, wherein:
the at least one pressure sensor includes one or more of a withdrawal pressure sensor or an infusion pressure sensor;
the withdrawal pressure sensor is configured for coupling with the withdrawal line to sense a withdrawal pressure signal indicative of a pressure in the withdrawal line;
the infusion pressure sensor is configured for coupling with the infusion line to sense an infusion pressure signal indicative of a pressure in the infusion line; and
the one or more pressure signals includes at least one of the withdrawal pressure signal or the infusion pressure signal.

7. The blood filtration system of claim 5, further comprising:
a blood pump configured to engage the withdrawal line to pump the blood through the withdrawal line and into the filter at an extracorporeal blood flow rate; and
a filtration pump configured to remove a filtrate fluid including the removed portions of the one or more plasma constituents from the filter at an ultrafiltration rate.

8. The system of claim 1, further comprising a damping system, the damping system is configured to damp flow of fluid, the damping system including:
an external infusion line configured for fluidic communication with the infusion port;
a flow restrictor in fluidic communication with the external infusion line, the flow restrictor having an orifice to restrict flow through the external infusion line; and
a check-valve in fluidic communication with one or more of the flow restrictor or the external infusion line, wherein:
the check-valve is configured to permit flow of fluid through a valve body in a first direction; and
the check-valve is configured to restrict the flow of fluid through the valve body in a second direction.

9. The system of claim 8, further comprising a blood circuit pump in fluidic communication with the infusion port, and the circuit pump is configured to pump fluid in one or more of the external infusion line or the blood circuit.

10. The system of claim 9, wherein operation of one or more of the circuit pump or the external infusion pump generates variations in flow of fluid in one or more of the external infusion line or the blood circuit, and the damping system is configured to damp the variations in flow of fluid.

11. The system of claim 9, wherein the pump is a peristaltic pump.

12. The system of claim 9, further comprising a flow sensor in communication with one or more of the external infusion line or the blood circuit, and the flow sensor is configured to sense a flow rate signal indicative of flow rate within one or more of the external infusion line or the blood circuit.

13. The system of claim 12, wherein the damping system cooperates with the flow sensor to damp variations in the flow rate signal based on operation of one or more of the external infusion pump or the circuit pump.

14. The system of claim 12, wherein the damping system is located between the flow sensor and the infusion port.

15-52. (canceled)
